(19) United States
(12) Patent Application Publication
(10) Pub. No.: US 2014/0309293 A1
(43) Pub. Date: Oct. 16, 2014

(54) CRYSTALLINE FORM

(71) Applicants: Antonio Nardi, Segrate (MI) (IT); Annibale Salvi, Milano (IT); Flavio Villani, Milano (IT); Bruno De Angelis, Segrate (MI) (IT); Giorgio Bertolini, Segrate (MI) (IT)

(72) Inventors: Antonio Nardi, Segrate (MI) (IT); Annibale Salvi, Milano (IT); Flavio Villani, Milano (IT); Bruno De Angelis, Segrate (MI) (IT); Giorgio Bertolini, Segrate (MI) (IT)

(73) Assignee: LABORATORIO CHIMICO INTERNAZIONALE S.P. A., Milano (IT)

(21) Appl. No.: 14/131,746

(22) PCT Filed: Jul. 6, 2012
(86) PCT No.: PCT/IB2012/001343

§ 371 Date: Apr. 15, 2014

(30) Foreign Application Priority Data
Jul. 11, 2011 (IT) MI2011A001286

(51) Int. Cl. C07D 339/04; A61K 31/381

(52) U.S. Cl. CPC C07D 339/04; A61K 31/381
USPC 514/440; 549/39

(57) ABSTRACT

The invention concerns a new polymorphic form of R-(+)-α-lipoic acid and a process for the preparation thereof, in addition to the compositions that contain it and its use as a medicine or food supplement.

### SUMMARY OF THE INVENTION

The present invention concerns a new polymorphic form of R-(+)-α-lipoic acid and a process for the preparation thereof, in addition to the compositions that contain it and its use as a medicine or food supplement.

### TECHNICAL BACKGROUND

R-(+)-α-lipoic acid (or (R)-5-(1,2-dithiolan-3-yl)pentanoic acid) is a liposoluble compound, widely used as a drug and as a food supplement mainly due to its antioxidant properties and its activity as a coenzyme in cell metabolism reactions. R-(+)-α-lipoic acid is chemically unstable and tends to easily polymerise, with consequent reduction in its bioavailability and beneficial properties.

It is known that the behaviour of substances taken as drugs and food supplements is determined by the chemical structure of said substances.

Different crystals of the same molecule, i.e. having a different crystalline structure, are defined polymorphic. Said differences generally lead to modification of the chemical-physical characteristics of the substance and said modifications in turn reflect on the pharmacological and pharmacokinetic properties of the molecule. The polymorphs of the same molecule can therefore have different stability, density, solubility and chemical reactivity.

The discovery of new crystalline forms of pharmaceutically useful compounds therefore provides new opportunities for improving the beneficial properties of said compounds.

R-(+)-α-lipoic acid has been described in various crystalline forms.

U.S. Pat. No. 5,455,264 describes a different crystalline form of the acid which is obtained by slow crystallisation from a mixture of cyclohexaneethyl acetate in a ratio of 2:1 or 5:1.

U.S. Pat. No. 6,44,1024 describes a different crystalline form of R-(+)-α-lipoic acid which is prepared by slow crystallisation in a toluenehexane or tolueneheptane mixture.

### OBJECT OF THE INVENTION

The object of the present invention is to provide a new crystalline form (otherwise also referred to as “polymorphic form” or “polymorph”) of R-(+)-α-lipoic acid which is both stable and bioavailable.

### DISCLOSURE OF THE INVENTION

According to one of its aspects, the invention concerns a crystalline form of R-(+)-α-lipoic acid having the following crystalline cell characteristics determined by X ray on the single crystal:

Monoclinic crystal, P2₁ a=5.564 (1) Å
b=9.793(1) Å
c=18.248(2) Å
β=91.776(6) Å
V=994 (1) Å³
D_{calc}=1.379 gcm⁻³

According to a preferred embodiment, the crystalline form of the R-(+)-α-lipoic acid of the present invention has the following characteristic 2-Theta peaks by X ray diffractometric analysis: 18.1, 20.6, 23.2 and 24.8.

According to a preferred embodiment, the crystalline form of the R-(+)-α-lipoic acid of the present invention has the X ray pattern of FIG. 1.

The crystalline form of the present invention has a good thermodynamic stability and therefore offers many advantages.

By accelerated stability test it was ascertained that the new crystalline form is more stable than the known crystalline forms. Since it is a pharmaceutically active molecule which is notoriously very reactive and tends to polymerise, improved stability obviously represents a very important quality.

### BRIEF DESCRIPTION OF THE DRAWINGS

FIG. 1 shows the X ray diffraction spectrum of the new crystalline form of the R-(+)-α-lipoic acid.

The new crystalline form of the invention is obtained with high purity by means of the simple procedure described below.

According to another of its embodiments, the invention concerns a process for preparation of the crystalline form of the R-(+)-α-lipoic acid of the invention, which comprises the following steps:
a. dissolving R-(+)-α-lipoic acid in a cyclohexane/ethyl acetate mixture by heating;
b. optionally filtering the solution of step (a);
c. rapidly cooling the solution thus obtained to 5-10° C., obtaining a suspension; and
d. optionally, isolating the crystalline form thus obtained.

According to a preferred embodiment, the cyclohexane/ethyl acetate mixture is in a ratio equal to or greater than 15/1, advantageously equal to or greater than 18/1.

The R-(+)-α-lipoic acid/solvent mixture ratio is not critical and the only conditions are that all the R-(+)-α-lipoic acid dissolves in said mixture when hot and that the quantity of solvent mixture is not able to maintain all the R-(+)-α-lipoic acid dissolved also when cold. By way of example, a ratio of between 1/5 and 1/10 (g/ml) can be used, for example around 1/6 or 1/7 (g/ml).

The solution of step (a) is prepared by heating for example up to approximately 35-40° C., advantageously 36-38° C., after which the solution is hot-filtered if necessary and then rapidly cooled to precipitate the new crystalline form.

The expression “rapidly cooled” indicates cooling with the aid of external means and not simply gradual cooling obtained by leaving the solution to slowly return to ambient temperature. Said rapid cooling can be obtained, for example, by placing the reaction receptacle in a blast chiller or in a bath of ice or water/ice or similar. The rapid cooling of the solution in step (c) is advantageously performed in a period of time between 10 and 60 minutes, advantageously between 15 minutes and 30 minutes.

The suspension obtained in step (c) can then be left under stirring at a temperature of 5-10° C. for a few hours.

The precipitate can then be isolated according to the common techniques, known to a person skilled in the art, for example by filtering.

It has been noted, unexpectedly and surprisingly, that rapid cooling of the solution containing the R-(+)-α-lipoic acid allows formation of the new crystalline form and that said polymorph is particularly stable with respect to the known polymorphs of the R-(+)-α-lipoic acid.

Further details of the process described above are provided in the experimental section of the present description.

According to another of its aspects, the invention concerns a pharmaceuticalnutraceutical composition comprising, as active ingredient, the crystalline form of the R-(+)-α-lipoic acid, if necessary in combination with one or more pharmaceutically acceptable carriers and/or excipients.

The composition of the invention can be formulated according to the methods well known in the art, for example it can be formulated in dosage units or in a multi-dose form.

The appropriate forms of administration preferably comprise oral administration such as tablets, capsules, powders, granules, and oral solutions or suspensions, sublingual and buccal forms of administration.

Capsules, preferably gelatin capsules, are preferred forms.

Gastro-resistant and/or prolonged release forms can also be prepared.

Advantageous forms include the pellets of R-(+)-α-lipoic acid described in the patent application WO2007/138022.

Other useful forms of administration are the dosage forms suitable for topical and/or transdermal administration, for example for use of the R-(+)-α-lipoic acid as an anti-ageing agent and/or to reduce the damage caused by exposure to UV rays. The preparation of these forms of administration is well known to a person skilled in pharmaceutical techniques.

In the composition of the invention, the new crystalline form of the R-(+)-α-lipoic acid can be formulated in combination with other active ingredients.

According to another embodiment, the invention concerns use of the new crystalline form of the R-(+)-α-lipoic acid for the preparation of a medicament and/or a food supplement.

The new crystalline form of the R-(+)-α-lipoic acid for use as an antioxidant and in the treatment of disorders connected with oxidative conditions and/or deriving from diseases of degenerative origin, for example diabetes, arteriopathies, retinopathies, central neuropathies, peripheral neuropathies, cerebral ageing, senile debilitation, hepatopathies, etc., represents a further aspect of the invention.

The invention also concerns a method for the treatment of disorders connected with oxidative conditions and/or deriving from diseases of degenerative origin, for example diabetes, arteriopathies, retinopathies, central neuropathies, peripheral neuropathies, cerebral ageing, senile debilitation, hepatopathies, etc., which comprises administering to a subject requiring it, advantageously to a mammal, such as man, an effective quantity of the new crystalline form of the R-(+)-α-lipoic acid of the invention.

The following examples illustrate the invention in a non-limiting manner.

### EXPERIMENTAL SECTION

### Example 1

### Preparation of the New Crystalline Form of R-(+)-α-Lipoic Acid

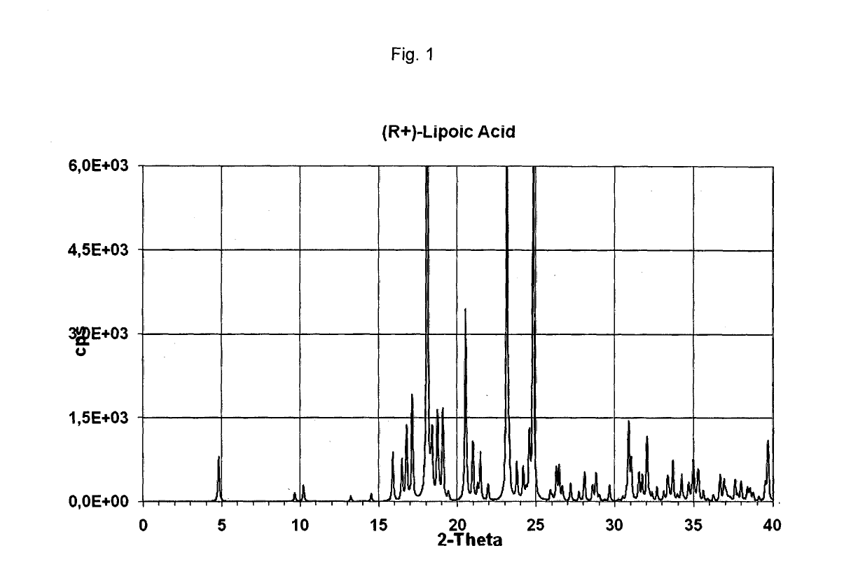

200 g of R-(+)-α-lipoic acid are dissolved in a mixture of cyclohexane (1279 ml) and ethyl acetate (70 ml), at 35-38° C. The solution thus obtained is filtered and cooled rapidly to 5-10° C. in 15 minutes. The suspension thus obtained is stirred at 5-10° C. for 2 hours and then filtered, washed with cyclohexane (70 ml) and dried in a vacuum at 20-25° C. to give 130 g of crystalline product, the X ray pattern of which is given in FIG. 1.

1. Crystalline form of R-(+)-α-lipoic acid having the following crystalline cell features by X ray on the single crystal:
Monoclinic Crystal, P2₁ a=5.564 (1) Å
b=9.793(1) Å
c=18.248(2) Å
β=91.776(6) Å
V=994 (1) Å³
D_{calc}=1.379 gcm⁻³

2. Crystalline form of R-(+)-α-lipoic acid having the following characteristic 2-Theta peaks by X ray diffractometric analysis 18.1, 20.6, 23.2 e 24.8.

3. The crystalline form according to claim 2, having the X ray pattern of FIG. 1.

4. A process for the preparation of the crystalline form of R-(+)-α-lipoic acid as defined in claim 1, which comprises the following steps:
(a) dissolving R-(+)-α-lipoic acid in a mixture cyclohexaneethyl acetate by heating;
(b) optionally filtering the solution of step (a);
(c) cooling rapidly the solution so obtained to 5-10° C., obtaining a suspension;
(d) optionally, isolating the crystalline form thus obtained.

5. The process according to claim 4, wherein said mixture cyclohexane/ethyl acetate has a ratio equal to 15/1 or higher.

6. The process according to claim 4, wherein the solution of step (a) is prepared by heating to approx. 35-40° C.

7. The process according to claim 4, wherein the suspension obtained in step (c) is stirred at 5-10° C.

8. A pharmaceutical/nutraceutical composition comprising, as the active ingredient, the crystalline form of R-(+)-α-lipoic acid as defined in claim 1, optionally in combination with one or more pharmaceutically acceptable carriers and/or excipients.

9. The composition of claim 8 which also comprises one or more further active ingredients.

10. Crystalline form of R-(+)-α-lipoic acid as defined in claim 1, for use as antioxidant and in the treatment of impairments linked to oxidative conditions and/or deriving from neurodegenerative diseases.
